(19) United States
(12) Patent Application Publication
(10) Pub. No.: US 2026/0048173 A1
(43) Pub. Date: Feb. 19, 2026

(54) ECO-SKIN: A Kombucha SCOBY-Derived Cellulose Matrix for Accelerated Healing and Antimicrobial Defense

(71) Applicant: Jonah Goodman, New York, NY (US)

(72) Inventor: Jonah Goodman, New York, NY (US)

(21) Appl. No.: 19/297,806

(22) Filed: Aug. 12, 2025

Related U.S. Application Data
(60) Provisional application No. 63/683,180, filed on Aug. 14, 2024.

(51) Int. Cl. A61L 26/00; A61K 31/121; A61K 31/353; A61K 35/74; A61K 36/82; A61P 17/02; A61P 31/04

(52) U.S. Cl. CPC A61L 26/0057; A61K 31/121; A61K 31/353; A61K 35/74; A61K 36/82; A61L 26/0066; A61L 26/0095; A61P 17/02; A61P 31/04; A61L 2300/30

(57) ABSTRACT

A reactivatable wound dressing composition comprising a dehydrated bacterial cellulose matrix derived from kombucha fermentation of organic green tea and Manuka honey. The matrix contains embedded dormant colonies of acetic acid bacteria, residual epigallocatechin gallate from green tea, and methylglyoxal from Manuka honey. The dormant bacterial colonies reactivate upon contact with an aqueous kombucha solution, providing antimicrobial activity against pathogens including Staphylococcus aureus, Escherichia coli, and Pseudomonas aeruginosa. The dehydrated matrix exhibits natural adhesive properties upon rehydration without requiring additional adhesives. Manufacturing methods involve controlled fermentation at specific pH and temperature parameters, followed by dehydration to preserve bacterial viability. The dressing maintains optimal wound environments by retaining moisture while allowing oxygen exchange, stimulates tissue regeneration, and can be die-cut into various anatomical shapes. Applications include treatment of cuts, burns, surgical incisions, chronic wounds, and skin ulcers with extended wear times of 5-10 hours.

### CROSS REFERENCE TO RELATED APPLICATIONS

The present application claims the priority benefit of U.S. Provisional Patent Application Ser. No. 63/683,180 filed on Aug. 14, 2024, which is hereby incorporated by reference in its entirety.

### TECHNICAL FIELD

The present disclosure relates to wound care compositions and methods, particularly to bacterial cellulose-based wound dressings derived from kombucha fermentation that provide antimicrobial protection and promote accelerated wound healing.

### BACKGROUND

Wounds are injuries which occur when sudden interference such as abrasions, burns, surgical incisions, cuts, or rashes occur to the surface of the skin, ranging from cosmetic/superficial damages to deep tissue destruction. As with all wounds, the primary concern is the chance of infection/chronicity (the likelihood of a wound becoming necrotic, infected, or delayed in healthy tissue formation). Chronic wounds, skin ulcers, and other wound related complications affect approximately 6.5 million patients in the United States annually, and as many as 37 million globally. The cost estimates for managing these wounds range from $28.1 to $96.8 billion annually. As a result, wounds pose a significant challenge in healthcare, often referred to as a “silent epidemic.” And without consistent and effective treatment, wounds, both chronic and acute, can lead to complications such as infection, tissue decay, delayed healing and an overall diminished quality of life. Therefore, timely interventions and comprehensive wound care treatments are essential to prevent complications, streamline the healing process, and improve patient outcomes.

### BRIEF SUMMARY OF EXEMPLARY EMBODIMENTS

The present disclosure provides exemplary embodiments directed to reactivatable wound dressing compositions and associated methods for their manufacture, activation, and therapeutic use.

### Reactivatable Wound Dressing Composition

Exemplary embodiments encompass a reactivatable wound dressing composition comprising a dehydrated bacterial cellulose matrix derived from kombucha fermentation of organic green tea and Manuka honey. The matrix comprises bacterial cellulose fibers having dormant colonies of acetic acid bacteria embedded therein, residual epigallocatechin gallate from the organic green tea, and residual methylglyoxal from the Manuka honey. The dormant colonies are configured to reactivate upon contact with an aqueous solution such as sterile water, saline solution, fermented kombucha, and diluted Manuka honey solution to provide antimicrobial activity, and the dehydrated matrix exhibits adhesive properties upon rehydration without requiring additional adhesives.

Various embodiments include compositions having moisture contents of 10-12% in the dehydrated state, and die-cut shapes including squares, rectangles, circles, ellipses, strips, and anatomically contoured shapes configured to conform to specific body regions, including but not limited to knees, elbows, heels, knuckles, and fingertips. Additional embodiments incorporate methylglyoxal derived from Manuka honey having UMF ratings of 10 or greater and maintain tensile strength sufficient to preserve structural integrity when hydrated.

### Manufacturing Methods

Exemplary embodiments includes a method for making a reactivatable bacterial cellulose wound dressing, comprising brewing organic green tea in distilled water at a concentration of 0.5% to 1.0% (w/v), cooling the brewed tea solution to a temperature between 25° C. and 30° C., adding medical-grade Manuka honey having a UMF rating of 10+ or greater to the cooled tea solution at a concentration of 8% to 12% by weight/volume, adding a SCOBY starter culture comprising fermented kombucha at a concentration of 10% to 20% by weight/volume, fermenting the mixture under static, aerobic conditions at 28° C.±2° C. Harvest when the bacterial cellulose (BC) concurrently satisfies three criteria: (1) pH 3.5-4.0, (2) acetic acid 0.5-1% and (3) thickness of the bacterial cellulose is approximately ¼^{th} in. Once these criteria are met, the bacterial cellulose is removed, dehydrated at a temperature of 30° C. to 40° C. for 5-7 hours to create dormant colonies of bacteria while maintaining their ability to reactivate upon subsequent hydration, wherein the dehydrated pellicle retains 10-12% moisture content. The resulting pellicle is die cut into specific shapes depending on the desired use case.

### Activation Methods

Exemplary embodiments encompass methods for activating dehydrated bacterial cellulose by means of a dual-chamber blister activation pack containing a dehydrated ECO-SKIN wound dressing and a pre-measured rehydration solution comprising but not limited to fermented manuka honey kombucha activation solution, saline, buffered saline, bioactive honey solutions, acetic acid solutions, antiseptic solutions, vitamin solutions, antibiotic solutions, pH-adjusted solutions, or combinations thereof. The activation solutions rehydrate cellulose fibers, reactivates dormant bacterial colonies, and generates adhesive properties through surface tension and residual Manuka honey tackiness. Various embodiments employ rehydration solutions selected from sterile water, saline solution, fermented kombucha, and diluted Manuka honey solutions.

### Treatment Methods

Exemplary embodiments include methods for treating wounds by applying the reactivatable compositions to wound sites, where the dressings maintain moist wound environments while providing antimicrobial protection. The methods encompass treatment of cuts, burns, surgical incisions, chronic wounds, and skin ulcers, with dressings remaining in place up to 10 hours before replacement. The antimicrobial protection targets bacteria including Staphylococcus aureus, Escherichia coli, and Pseudomonas aeruginosa.

### Wound Healing Enhancement

Exemplary embodiments provide methods for promoting wound healing where the bacterial cellulose matrix stimulates migration and proliferation of fibroblasts and endothelial cells, maintains optimal oxygen exchange while retaining moisture, provides semi-permeable barriers against contaminants, and creates acidic microenvironments that inhibit pathogenic bacterial growth. Various embodiments accelerate wound healing compared to conventional gauze dressings and reduce scarring formation.

### Sustained Antimicrobial Activity

Exemplary embodiments encompass methods for providing sustained antimicrobial activity at wound sites, where embedded acetic acid bacteria provide continuous antimicrobial activity through production of acetic acid and maintenance of acidic pH environments hostile to pathogenic microorganisms.

Exemplary embodiments encompass methods for providing sustained antimicrobial activity at wound sites, where rehydration solutions provide antimicrobial activity through environments hostile to pathogenic microorganisms.

These exemplary embodiments provide comprehensive wound care solutions that combine controlled fermentation processes with bioactive materials to address infection control, optimal healing environments, and patient comfort while offering advantages over conventional wound dressing approaches.

### BRIEF DESCRIPTION OF THE DRAWINGS: NO COLOR

FIG. 1: NO COLOR-FIG. 1 is a “wrap” embodiment of ECO-SKIN. The wrap is intended to fit the circumference of the arm, leg, or hand. The ECO-SKIN wrap is designed for large scale wounds, with a large emphasis on burns and post-surgical sites. This image is in black and white

FIG. 2: NO COLOR-FIG. 2 are various ECO-SKIN die cut shapes, with variations in shapes and sizes for different body parts and various wound applications. This image is in black and white

FIG. 3: NO COLOR-FIG. 3 is ECO-SKIN Strip in action, placed on the thumb, index and ring finger of the mannequin, as visual representation of the durability, rigidity and comfortability of ECO-SKIN. This image is in black and white

FIG. 4: NO COLOR-FIG. 4 is ECO-SKIN Wrap in action, placed on the wrist of the mannequin, as a visual representation of the durability, rigidity and comfortability of ECO-SKIN Wrap. This image is in black and white

FIG. 5: NO COLOR-FIG. 5 is ECO-SKIN Patch in action, and placed on the forearm of the mannequin, as a visual representation of the durability, rigidity and comfortability of ECO-SKIN Patch. This image is in black and white

FIG. 6: NO COLOR-FIG. 6 are the basic ingredients of ECO-SKIN. Organic Green Tea, Medical Grade Manuka honey UMF 10+, and an existing colony of acetic acid bacteria (Starter Culture). This image is in black and white

FIG. 7: NO COLOR-FIG. 7 is a workflow diagram, explaining the process of growing, harvesting and post processing ECO-SKIN. This image is in black and white

FIG. 8: NO COLOR-FIG. 8 illustrates a dual-chamber blister activation pack containing a dehydrated ECO-SKIN wound dressing (804) in one compartment and a pre-measured rehydration solution (802) in the other, separated by a breakable seal (803). Upon rupture of the seal, the solution flows into the dressing compartment to uniformly hydrate the bacterial cellulose matrix, ensuring precise liquid delivery, and optimal rehydration before application. This image is in black and white.

FIG. 9: COLOR-FIG. 9 is a workflow diagram, explaining the user sequence for rehydrating and applying using the dual-chamber blister activation pack. This image is in black and white.

FIG. 10: NO COLOR-FIG. 10 are various dehydrated ECO-SKIN shapes that have been vacuum sealed into sterile packaging for later use. The packaging also includes the lot number, batch number and size of the bandage. This image is in black and white

FIG. 11: NO COLOR-FIG. 11 are user sequence images for removing ECO-SKIN without an adhesive backing from the sterile dehydrated packaging and applying to skin without the use of the activation catalyst. This image is in black and white

FIG. 12: NO COLOR-FIG. 12 is a workflow diagram, for removing ECO-SKIN without an adhesive backing from the sterile dehydrated packaging and applying to skin without the use of the activation catalyst. This image is in black and white

FIG. 13: NO COLOR-FIG. 13 is a visual representations different configurations of ECO-SKIN shapes affixed to an adhesive backing, on a user's arm, without the use of the activation catalyst. This image is in black and white

FIG. 14: NO COLOR-FIG. 14 is a workflow diagram, for removing ECO-SKIN with an adhesive backing, from the sterile dehydrated packaging and applying to skin without the use of the activation catalyst. This image is in black and white

FIG. 15: NO COLOR-FIG. 15 are the results of Rehydrated ECO-SKIN Kirby Bauer antibacterial disk diffusion testing against Staphylococcus Aureus, Pseudomonas Fluorescence and Escherichia Coli. Clear zones of inhibition are shown indicating broad spectrum antibacterial properties. This image is in black and white

FIG. 16: NO COLOR-FIG. 16 are Scanning electron microscope images of a dehydrated ECO-SKIN showcasing varying levels of magnification: (100 μm, 500× Magnification), (500 μm, 100× Magnification), (50 μm, 1000× Magnification), (20 μm, 2000× Magnification). These SEM images showcase the porous nature of ECO-SKIN in addition to the imbedded acetic acid bacteria within the fiber network. This image is in black and white.

### BRIEF DESCRIPTION OF THE DRAWINGS: COLOR

The patent or application file contains at least one drawing executed in color. Copies of this patent or patent application publication with color drawing(s) will be provided by the Office upon request and payment of the necessary fee.

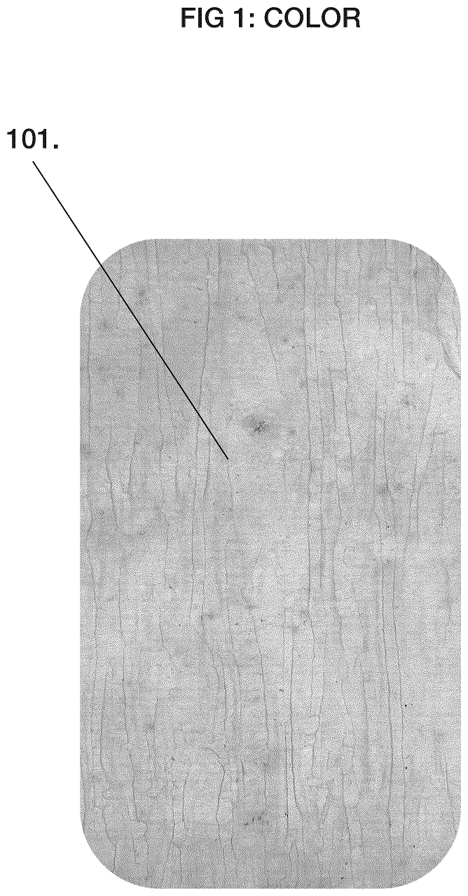

FIG. 1: COLOR-FIG. 1 is a “wrap” embodiment of ECO-SKIN. The wrap is intended to fit the circumference of the arm, leg, or hand. The ECO-SKIN wrap is designed for large scale wounds, with a large emphasis on burns and post-surgical sites. This image is in full RGB color.

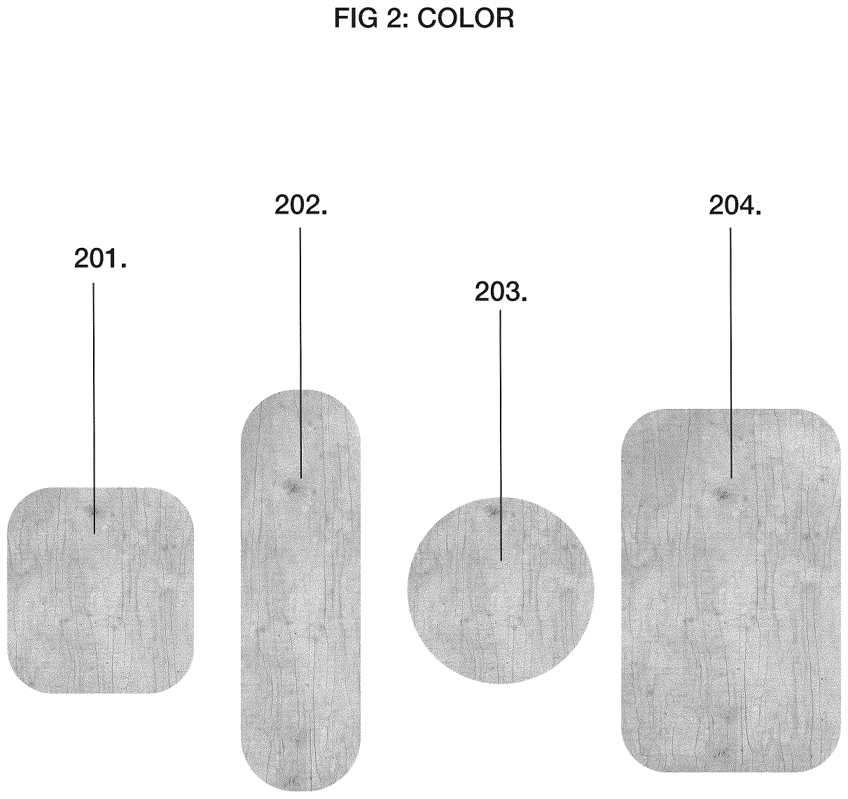

FIG. 2: COLOR-FIG. 2 are various ECO-SKIN die cut shapes, with variations in shapes and sizes for different body parts and various wound applications. This image is in full RGB color.

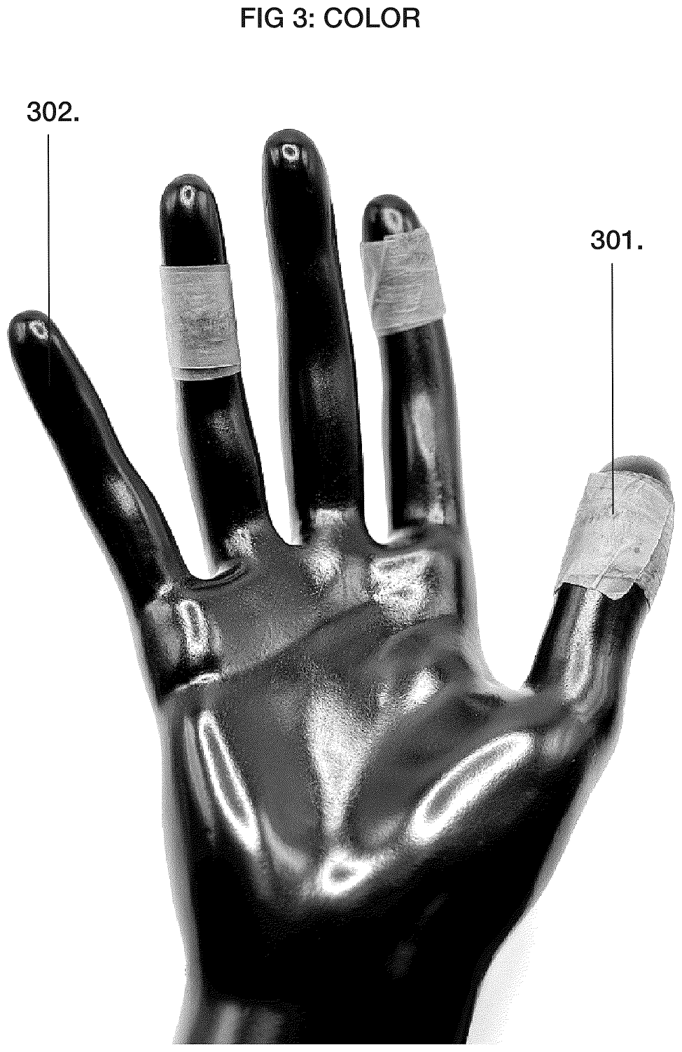

FIG. 3: COLOR-FIG. 3 is an ECO-SKIN Strip in action, placed on the thumb, index and ring finger of the mannequin, as visual representation of the durability, rigidity and comfortability of ECO-SKIN. This image is in full RGB color.

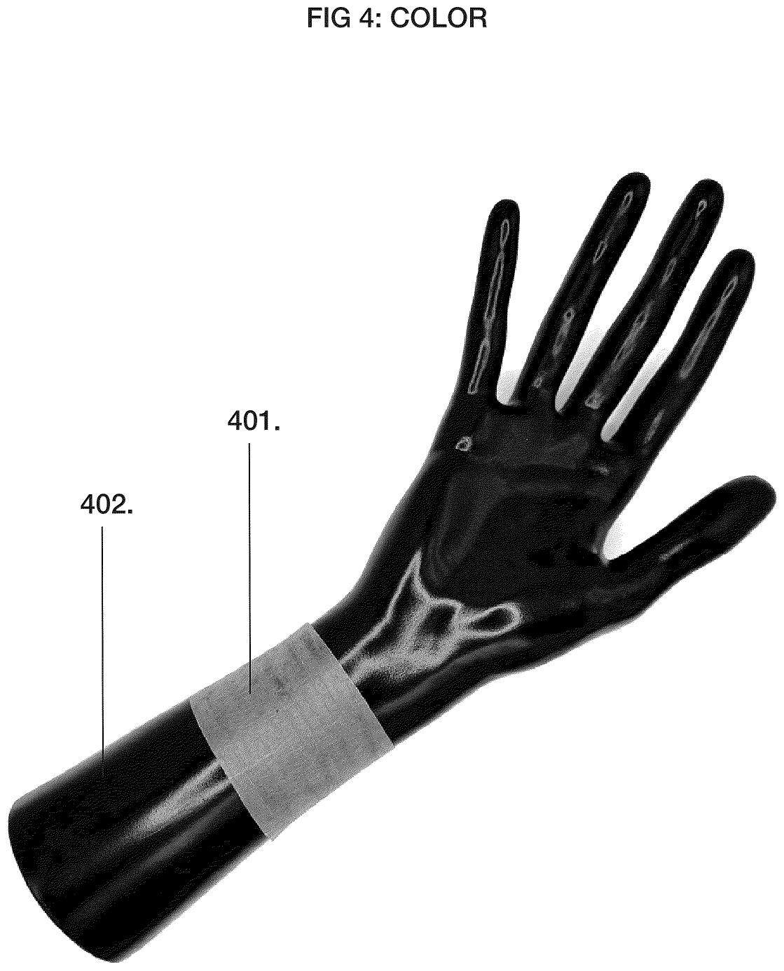

FIG. 4: COLOR-FIG. 4 is an ECO-SKIN Wrap in action, placed on the wrist of the mannequin, as a visual representation of the durability, rigidity and comfortability of ECO-SKIN Wrap. This image is in full RGB color.

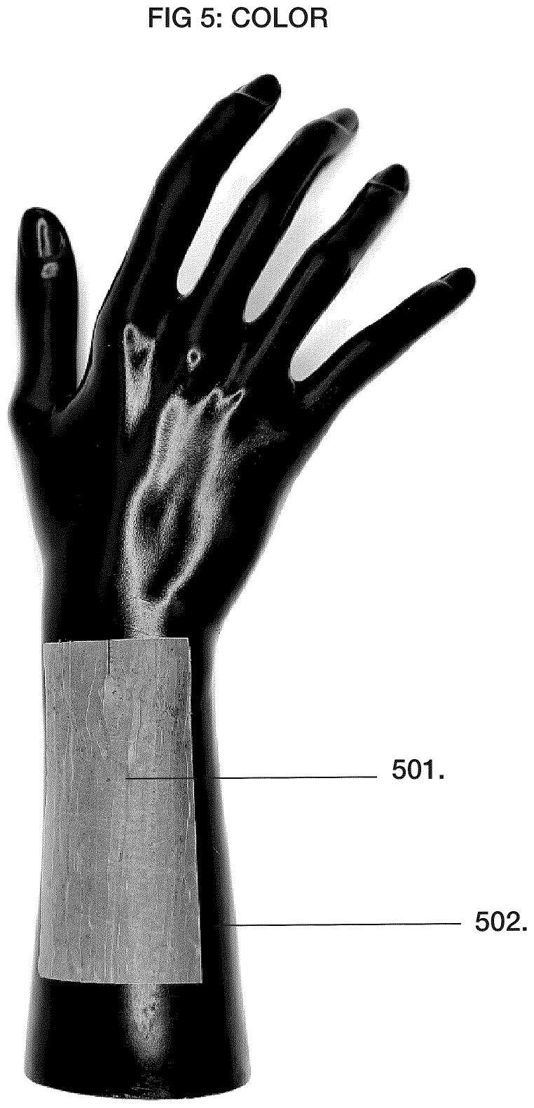

FIG. 5: COLOR-FIG. 5 is an ECO-SKIN Patch in action, and placed on the forearm of the mannequin, as a visual representation of the durability, rigidity and comfortability of ECO-SKIN Patch. This image is in full RGB color.

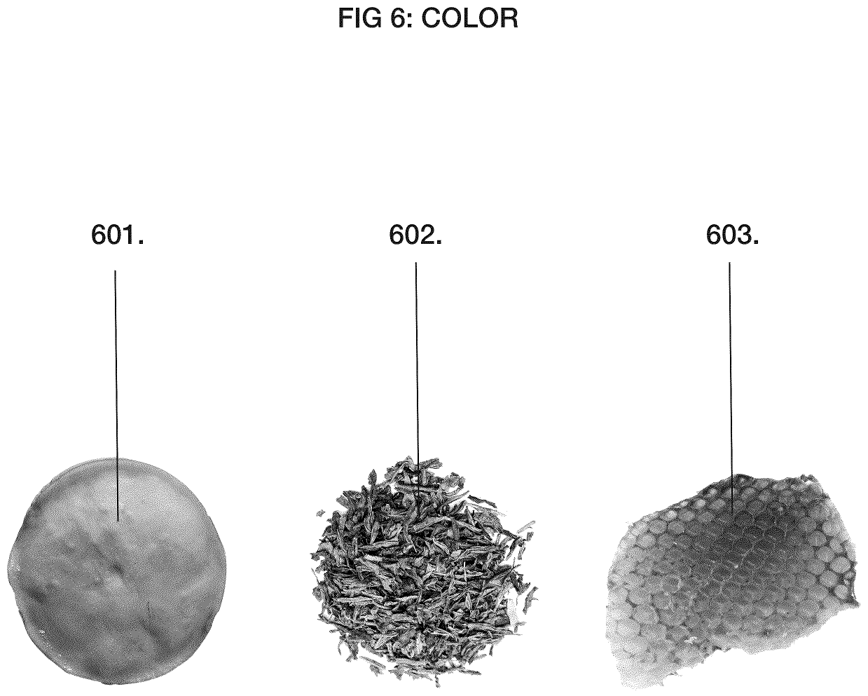

FIG. 6: COLOR-FIG. 6 are the basic ingredients of ECO-SKIN. Organic Green Tea, Medical Grade Manuka honey UMF 10+, and colony of acetic acid bacteria (Starter Culture). This image is in full RGB color.

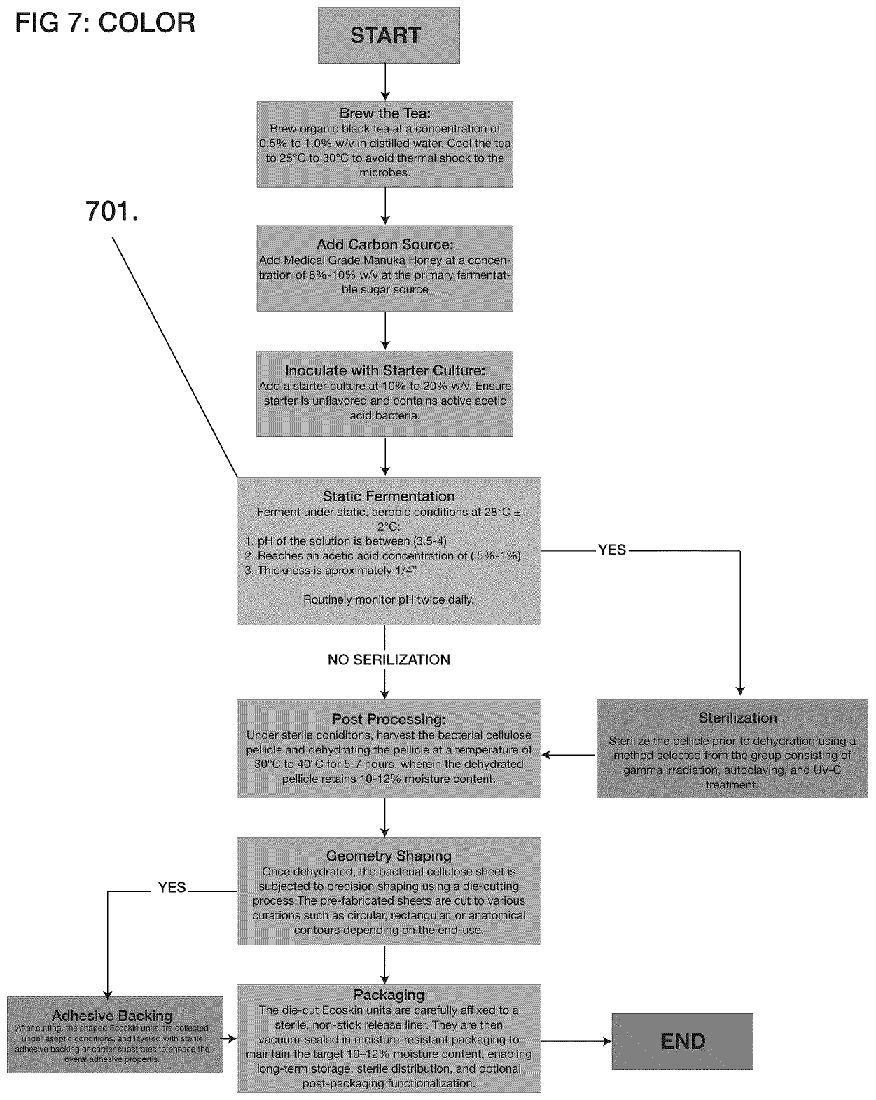

FIG. 7: COLOR-FIG. 7 is a workflow diagram, explaining the process of growing, harvesting and post processing ECO-SKIN. This image is in full RGB color.

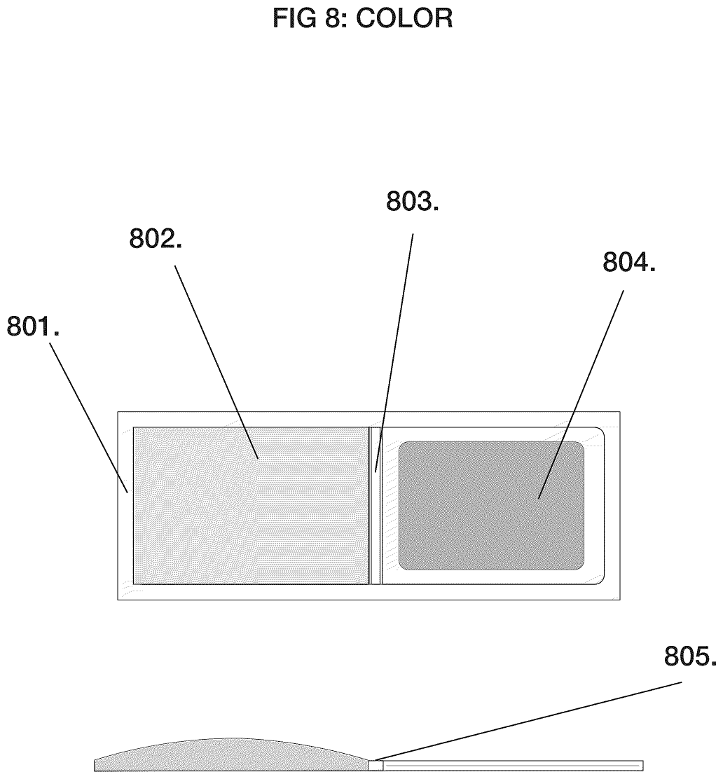

FIG. 8: COLOR-FIG. 8 illustrates a dual-chamber blister activation pack containing a dehydrated ECO-SKIN wound dressing (804) in one compartment and a pre-measured rehydration solution (802) in the other, separated by a breakable seal (803). Upon rupture of the seal, the solution flows into the dressing compartment to uniformly hydrate the bacterial cellulose matrix, ensuring precise liquid delivery, and optimal rehydration before application. This image is in full RGB color.

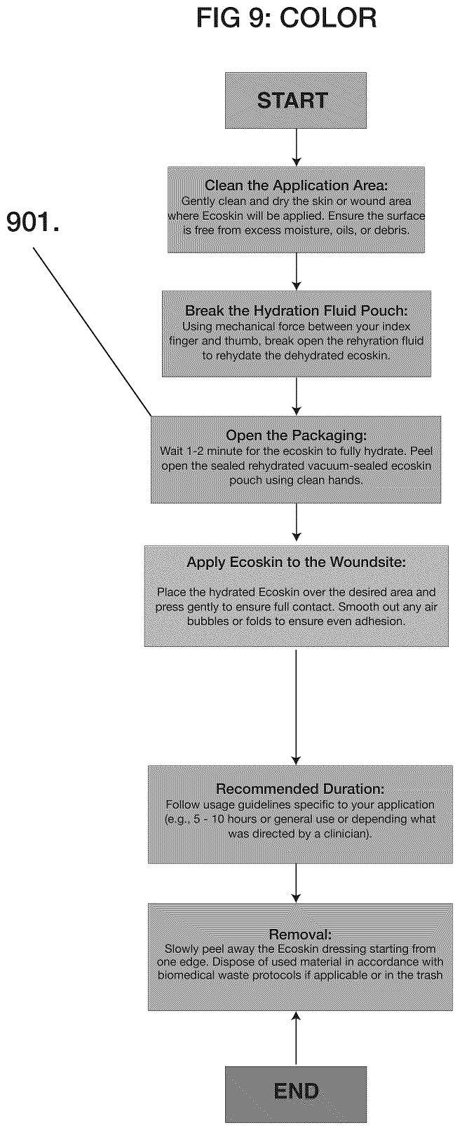

FIG. 9: COLOR-FIG. 9 is a workflow diagram, explaining the user sequence for rehydrating and applying using the dual-chamber blister activation pack. This image is in full RGB color.

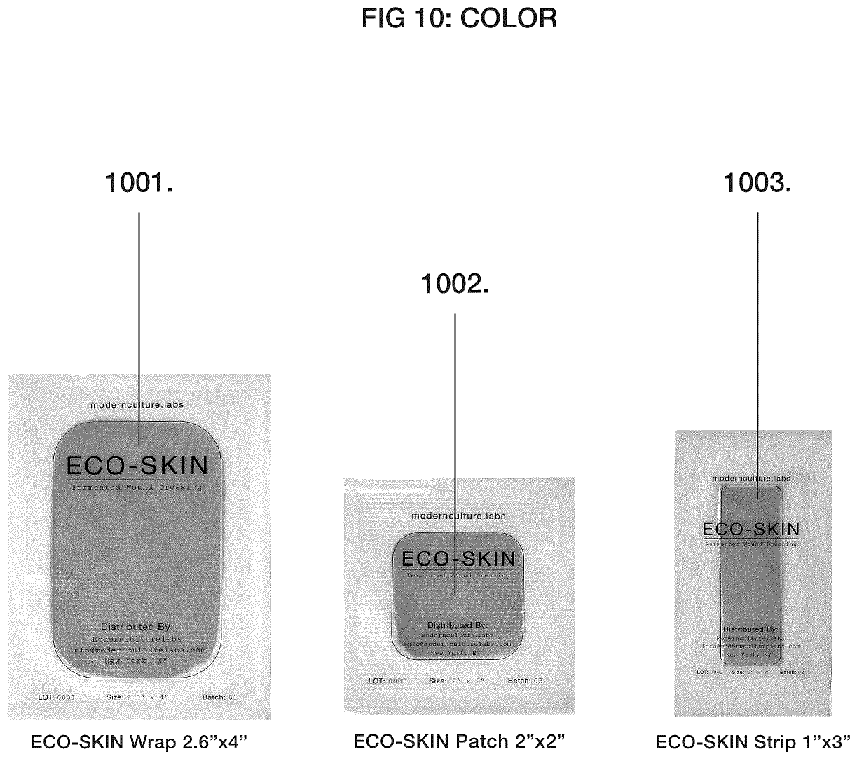

FIG. 10: COLOR-FIG. 10 are various dehydrated ECO-SKIN shapes that have been vacuum sealed into sterile packaging for later use. The packaging also includes the lot number, batch number and size of the bandage. This image is in full RBG color.

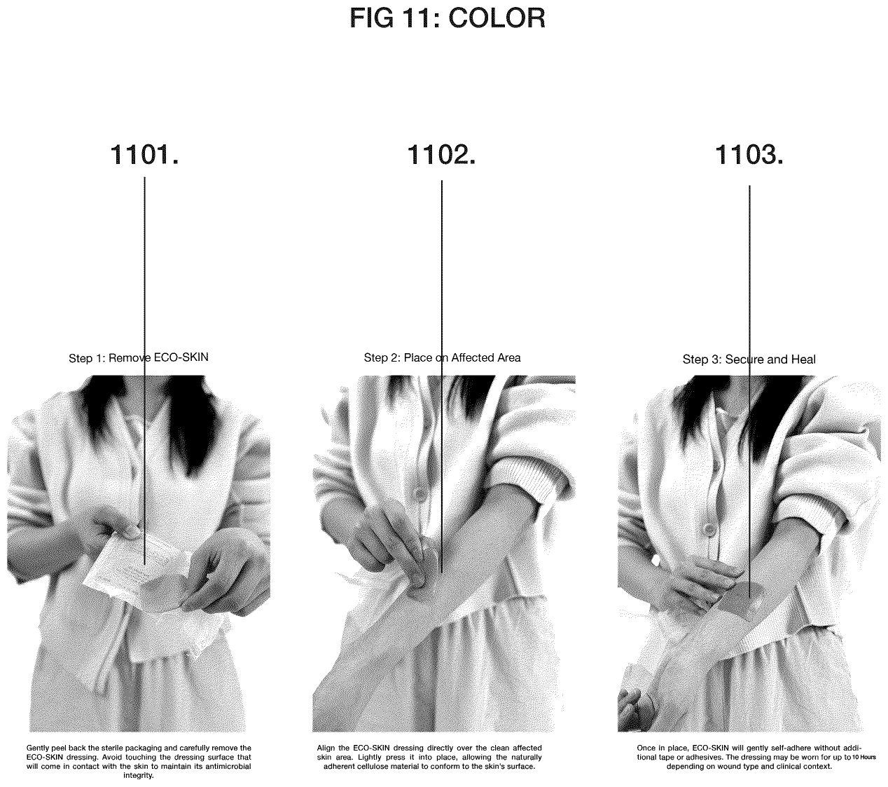

FIG. 11: COLOR-FIG. 11 are user sequence images for removing ECO-SKIN without an adhesive backing from the sterile dehydrated packaging and applying to skin without the use of the activation catalyst. This image is in full RBG color.

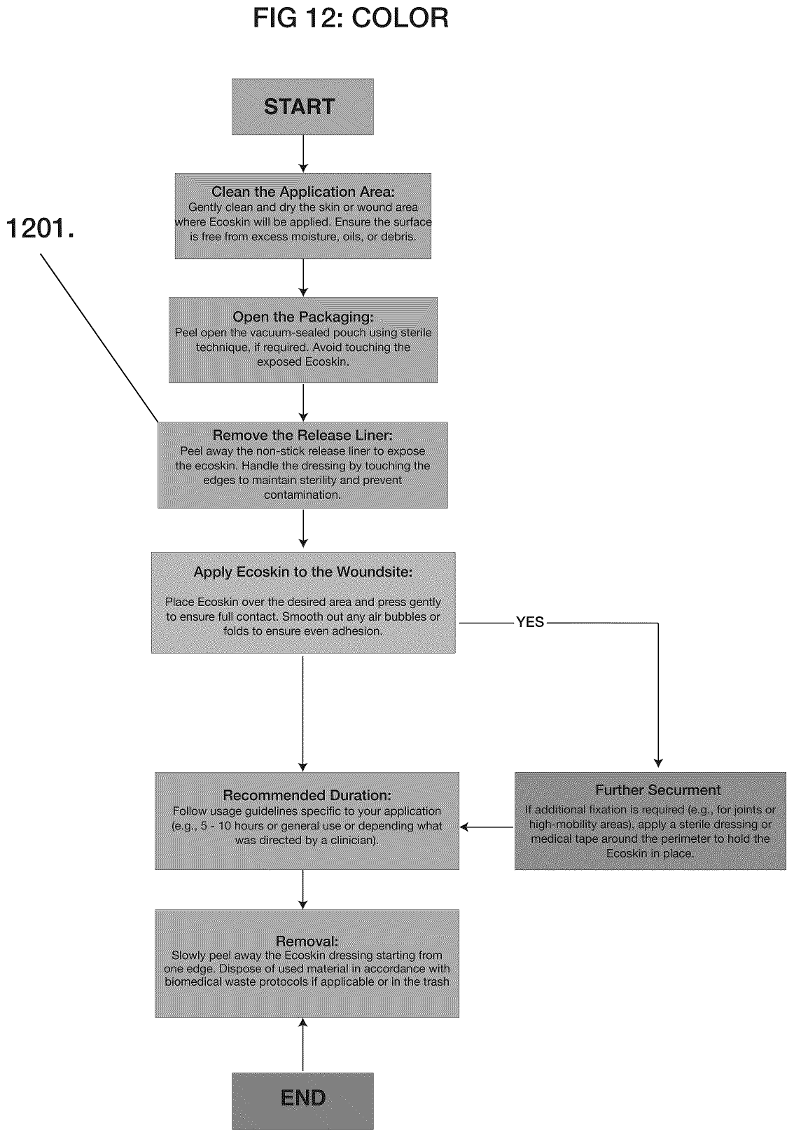

FIG. 12: COLOR-FIG. 12 is a workflow diagram, for removing ECO-SKIN without an adhesive backing from the sterile dehydrated packaging and applying to skin without the use of the activation catalyst. This image is in full RBG color.

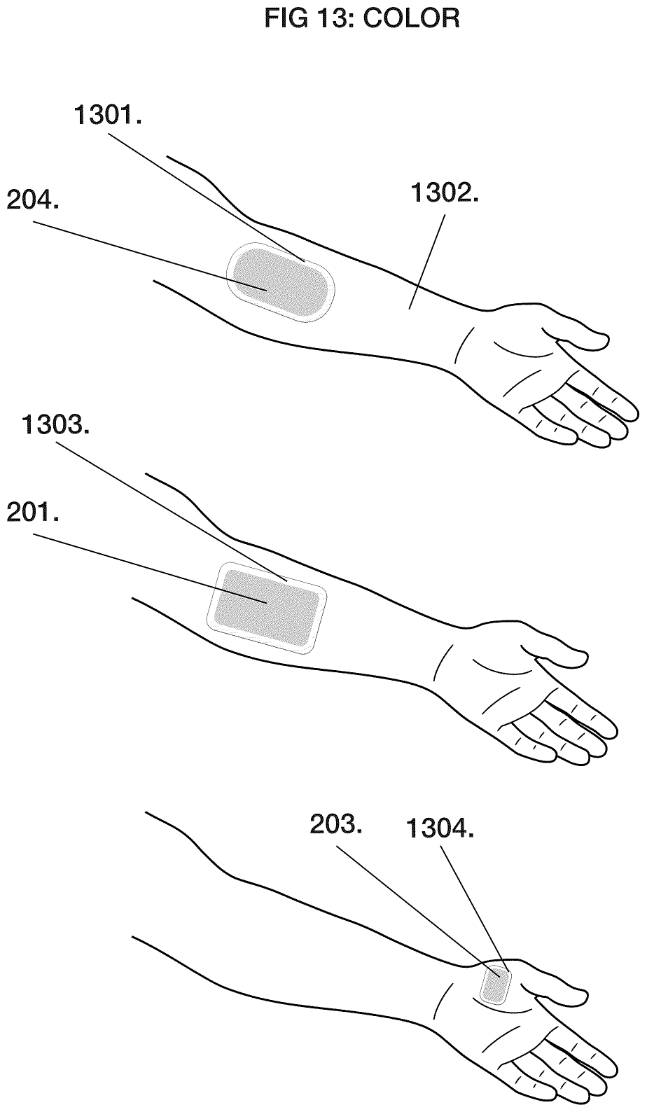

FIG. 13: COLOR-FIG. 13 is a visual representations different configurations of ECO-SKIN shapes affixed to an adhesive backing, on a user's arm, without the use of the activation catalyst. This image is in full RBG color.

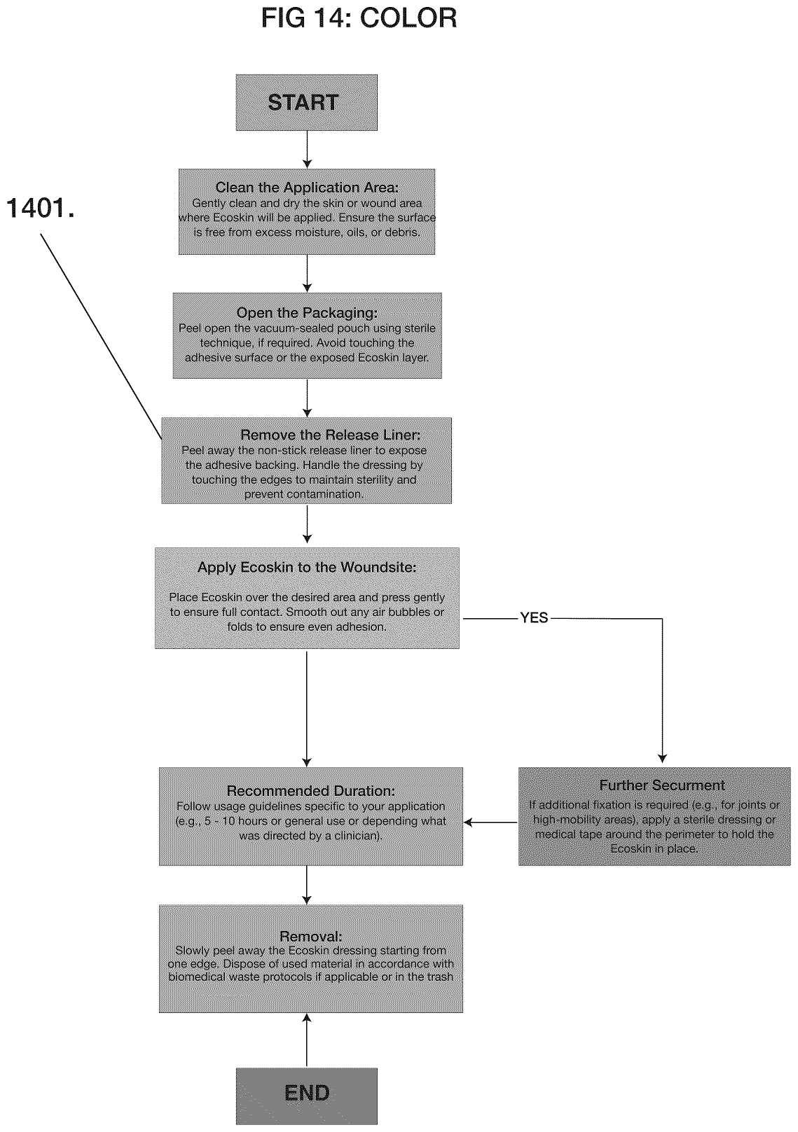

FIG. 14: COLOR-FIG. 14 is a workflow diagram, for removing ECO-SKIN with an adhesive backing, from the sterile dehydrated packaging and applying to skin without the use of the activation catalyst. This image is in full RBG color.

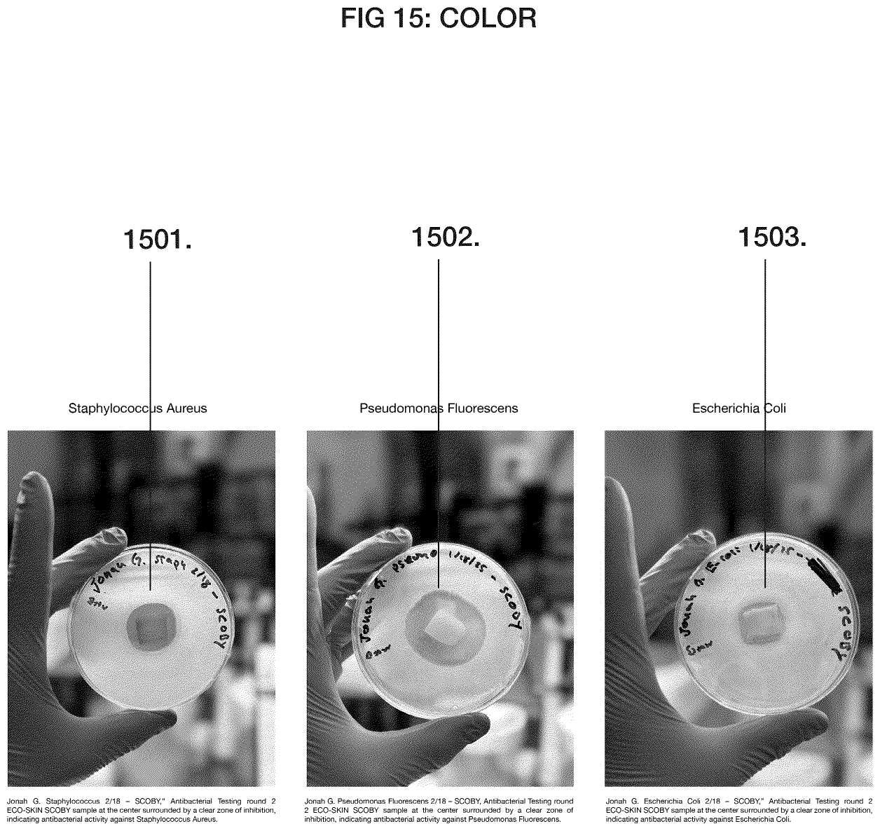

FIG. 15: COLOR-FIG. 15 are the results of Rehydrated ECO-SKIN Kirby Bauer antibacterial disk diffusion testing against Staphylococcus Aureus, Pseudomonas Fluorescence and Escherichia Coli. Clear zones of inhibition are shown indicating broad spectrum antibacterial properties. This image is in full RBG color.

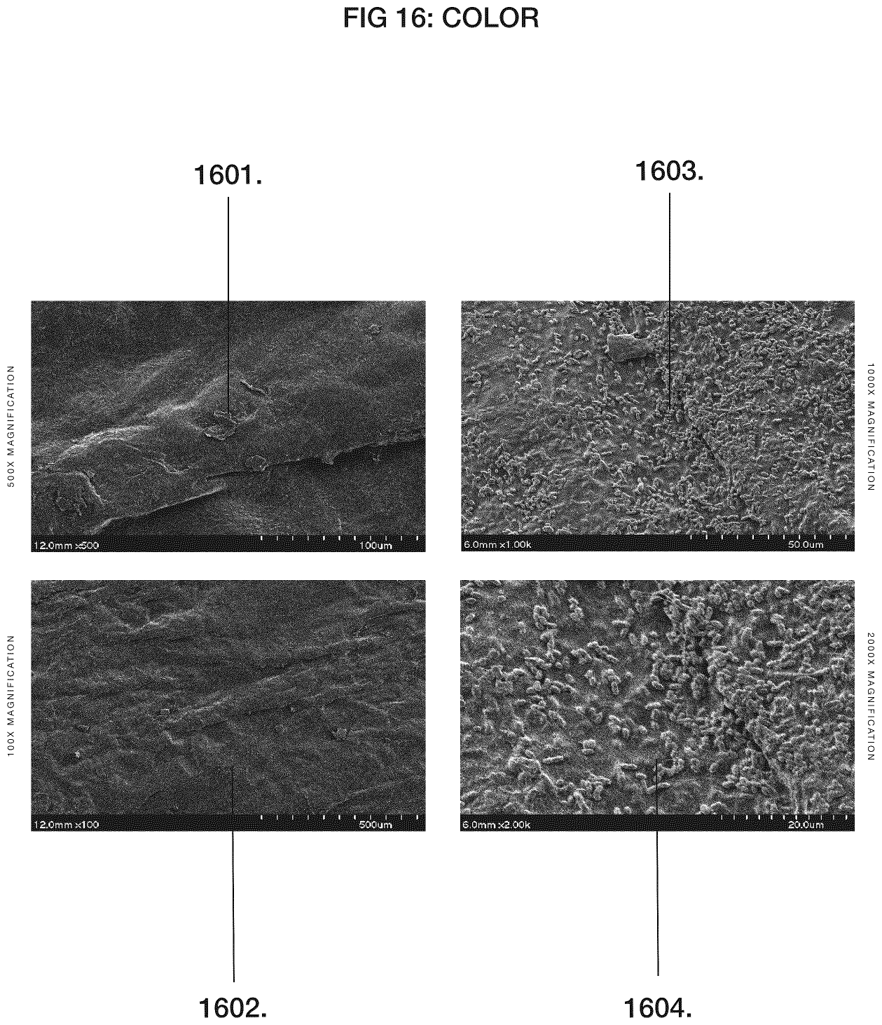

FIG. 16: COLOR-FIG. 16 are Scanning electron microscope images of a dehydrated ECO-SKIN showcasing varying levels of magnification: (100 μm, 500× Magnification), (500 μm, 100× Magnification), (50 μm, 1000× Magnification), (20 μm, 2000× Magnification). These SEM images showcase the porous nature of ECO-SKIN in addition to the imbedded acetic acid bacteria within the fiber network. This image is in full RBG color.

### REFERENCE NUMERALS

101. ECO-SKIN
201. ECO-SKIN Die Cut (Patch)
202. ECO-SKIN Die Cut (Strip)
203. ECO-SKIN Die Cut (Spot)
204. ECO-SKIN Die Cut (Wrap)
301. ECO-SKIN Strip (In Use)
302. Mannequin Fingers
401. ECO-SKIN Wrap (In Use)
402. Mannequin Wrist
501. ECO-SKIN Patch (In Use)
502. Mannequin Forearm
601. Starter Culture (SCOBY)
602. Organic Green Tea
603. Medical Grade Manuka Honey UMF 10+
701. ECO-SKIN Growth Process Flow Chart
801. Vacuum-Sealed Dual Chamber Blister Activation Pack: Breakable Seal
802. Vacuum-Sealed Dual Chamber Blister Activation Pack: Rehydration Solution
803. Vacuum-Sealed Dual Chamber Blister Activation Pack: Breakable Seal
804. Vacuum-Sealed Dual Chamber Blister Activation Pack: Dehydrated ECO-SKIN
805. Side View, Vacuum-Sealed Dual Chamber Blister Activation Pack
901. User Sequence, Dual Chamber Blister Activation Pack
1001. Vacuum Sealed ECO-SKIN Wrap
1002. Vacuum Sealed ECO-SKIN Patch
1003. Vacuum Sealed ECO-SKIN Strip
1101. User Opening Vacuum Sealed ECO-SKIN Patch
1102. User placing ECO-SKIN Patch on forearm
1103. User leaving ECO-SKIN Patch on forearm
1201. ECO-SKIN User Sequence with No Adhesive Backing/No Activation Catalyst
1301. ECO-SKIN Wrap Adhesive Backing
1302. Line Drawing of Mannequin Hand
1303. ECO-SKIN Patch Adhesive Backing
1304. ECO-SKIN Spot Adhesive Backing
1401. ECO-SKIN User Sequence with Adhesive Backing/No Activation Catalyst
1501. Kirby Bauer Disk Diffusion Test: Staphylococcus Aureus
1502. Kirby Bauer Disk Diffusion Test: Pseudomonas Fluorescence
1503. Kirby Bauer Disk Diffusion Test: Escherichia-Coli
1601. ECO-SKIN Scanning Electronic Microscope 100 μm, 500× Magnification
1602. ECO-SKIN Scanning Electronic Microscope 500 μm, 100× Magnification
1603. ECO-SKIN Scanning Electronic Microscope 50 μm, 1000× Magnification
1604. ECO-SKIN Scanning Electronic Microscope 20 μm, 2000× Magnification

### DETAILED DESCRIPTION OF EXEMPLARY EMBODIMENTS

FIG. 1: FIG. 1 is one exemplary embodiment of ECO-SKIN (101). ECO-SKIN is designed for large scale wounds or small-scale wounds, with an emphasis on burns, infected wound sites and post-surgical incisions. The range of factors affecting the wound healing process range from local factors (directly influence the characteristics of the wound itself), to systemic factors, (the baseline health of the individual), that affect their ability to heal properly. From newly found evidence, experts have identified key factors that directly affect the wound healing process: Proper oxygenation, maintaining of a moist wound environment, and prevent/reduce the onset of infection.

The colored image FIG. 1 COLOR: ECO-SKIN (101) offers a clearer and more detailed visual representation of the ECO-SKIN Patch, highlighting its structure, texture, and overall appearance. Colored images provide a higher level of detail compared to black-and-white images. For ECO-SKIN, this allows for a more precise depiction of the intricate structure, texture, and visual appearance of ECO-SKIN. This visual clarity can aid in better understanding the unique features and benefits of the product and have the users gain a sense of familiarity and visual presence.

### Oxygenation

Oxygenation is crucial for cell function, cell metabolism, energy production and for the wound healing process. During the inflammation stage of healing, the wound site becomes hypoxic and becomes deprived of oxygen. This occurs due to the disruption of vasculature surrounding the wound site, resulting in an increase in inflammatory cells to repair the damaged tissue and perform normal skin healing. However, prolonged hypoxia can cause tissue damage and further damage the wound site, which is the case of wounds that are covered for extended periods of time. In general, wounds should be covered periodically, but not all the time, to help them heal properly.

### Moist Wound Environment

Exposing a wound to air can dry it out, which can slow healing and increase the risk of infection. Covering a wound also helps maintain moisture, which keeps cells alive and can reduce the likelihood of scarring. As a result, a moist wound environment has been shown to promote a healthy wound process, by preventing the wound site from dehydration, which increases the rate of dead tissue buildup. Clinical studies have shown that a moist wound environment, under controlled hydration, stimulates keratinocyte proliferation and fibroblast growth, promoting healthy skin inflammation and healing success.

### Infection

When a wound becomes infected, the bacteria and germs can grow within the damaged skin, multiplying, and causing the wound site to delay proper healing. Often, these bacteria appear in the form of biofilms, which are a thick, slimy barrier of biopolymers, including polysaccharides, DNA, proteins, and lipids that appear on the wound site. They can form in either chronic or acute wounds, appear within 24 hours of a wound and are considered the main cause of healing delay. Furthermore, these biofilms help protect the bacteria from conventional antibiotic treatments. As a result, the emergence of Staphylococcus aureus (S. aureus), Pseudomonas aeruginosa (P. aeruginosa and β-hemolytic streptococci appear which are the most common types of bacteria that form from an infected wound site. Many of these biofilms are also antibiotic resistant due to the presence of biofilms containing P. Aeruginosa, which shields the bacteria from the antibiotic treatment.

Traditionally, in both hospital and home settings, wound dressing products like gauze, plasters, bandages, and cotton wool are used to protect wounds from contamination or abrasion. However, the intended purpose of gauze dressings is to protect the wound from external trauma and absorb wound fluid. This poses a significant problem. On their own, these dressings promote little breathability, offer low sterility or high moisture content for the wound to properly heal, and oftentimes require external agents such as antibacterial ointment in an attempt to slow the rate of infection and return the body to a state of homeostasis. Additionally, the need for frequent changes to prevent the formation of healthy tissues increases the chance of external contamination and infection. Promisingly, the development of innovative wound healing techniques, wraps and substrates, have been shown to improve wound healing time, reduce the frequency of changing dressings, and lower the incidence of complications.

Hydrogels, for example, are made from synthetic polymers that promote a moist wound environment, proper breathability and high durability, promoting healthy tissue granulation formation. They also enable the absorption and retention of wound excretion, which left on its own, has the potential to infect the wound site. Hydrocolloid patches, a hydrogel derivative, has seen a surge in popularity for wound healing and acne treatment. They're made from a combination of materials that form a gel when they come into contact with bodily fluids, such as pus. The gel creates a moist environment that helps wounds heal and protects new tissue. Alginate dressings, made from seaweed derivatives, additionally absorb wound extradites and promote healing by forming a protective film over the wound site.

Bioactive dressings are biocompatible and biodegradable that can help with healing by incorporating substances that have a therapeutic effect on the wound. They can be made from natural or synthetic materials and can impact the healing process directly or indirectly. They work by releasing bioactive factors or by using materials that have endogenous activity. They can create a more favorable environment for healing, unlike traditional dressings that mainly act as barriers against external contaminants. ECO-SKIN, being a bacterial cellulose dressing, is a natural polymer produced by the bacteria during kombucha fermentation and has been shown to be a highly efficacious wound dressing alternative for enhancing the outcomes of wounds.

Enhanced Moisture Retention and Absorption: One of the key advantages of bacterial cellulose (BC) is its ability to retain moisture while allowing for optimal oxygen exchange. This creates an ideal environment for wounds, by keeping the skin bed moist without causing excessive moisture retention. Additionally, bacterial cellulose can exudate and absorb excess fluid leaking from the wound site, maintain a clean and hygienic environment while reducing the risk of infection.

High Biocompatibility and Biodegradability: Bacterial cellulose is biocompatible, meaning it is well-tolerated by the body and does not elicit significant immune responses or adverse reactions. This makes it suitable for use on sensitive or compromised skin, as well as for long-term applications. BC is also biodegradable, breaking down naturally over time without leaving behind any harmful residues.

Structural Integrity and Conformability: The unique structure of bacterial cellulose gives it excellent mechanical properties, including high tensile strength, flexibility and adaptability. This allows bacterial cellulose to conform closely to the contours of the wound site, providing a contoured and comfortable fit that promotes optimal comfort without tugging on the skin during movement. Moreover, the structural integrity of bacterial cellulose ensures that it maintains its shape and integrity even when wet, minimizing the risk of fiber shedding and ensuring consistent wound coverage.

Promotion of Tissue Regeneration: Studies have shown that bacterial cellulose's bioactive properties, promote tissue regeneration and wound closure. This occurs because it stimulates the migration and proliferation of fibroblasts, endothelial, and other cells involved in the wound healing process.

Sustainability/Biodegradability: Bacterial Cellulose is grown from non-synthetic materials and avoids the use of synthetic additives or chemicals. This natural composition ensures that bacterial cellulose remains free from potentially toxic substances, making it safer for both patients and the planet. Moreover, bacterial cellulose is 100% biodegradable. This aligns with principles of sustainable resource management and reduces the burden on landfills and waste disposal systems. As bacterial cellulose decomposes, its fibrous properties provide nutrients for the soil, supporting ecological balance and soil health.

FIG. 2: FIG. 2 is the ECO-SKIN Die Cuts, with variations in shapes and sizes for different body parts and various wound applications. These designed can be customized and are derived using a steel die cutter. (201) is square shaped and is designed for general cuts, burns or scrapes. (203) Is circular and is designed for small scale cuts wounds that require circular bandages. (202) is oval shaped and is designed to fit the fingers/toes, mimicking the design of traditional Band-Aids. (204) is a large patch/wrap and is designed to fit large sections of the body high flexibility, such as legs and arms. ECO-SKIN's versatility and flexibility (201-204) allows it to conform to any surface on the skin, offering customizable shapes and contours tailored to the unique needs of each patient. ECO-SKIN has a smooth, paper-thin structure with high elasticity and durability. Unlike conventional dressings with rough edges, bulky fibers that thread or catch on clothing, ECO-SKIN provides a smooth surface ensuring comfort for the wearer. The waxy smooth finish of ECO-SKIN further enhances its usability, offering a gentle touch against the skin while maintaining secure coverage over the wound site.

The colored image FIG. 2 COLOR: ECO-SKIN (201-204) offers colored variety the various forms and shapes that ECO-SKIN can be customized into. The colored images can visually demonstrate this customization showing how the product can be tailored to different applications such as burns, cuts and surgical wounds. The color offers a higher level of visual detail, better depicting the structural and physical nuances between the different forms and shapes.

FIG. 3: FIG. 3 is the ECO-SKIN Strip configuration in action. ECO-SKIN Strip (301). is intended to function like Band-Aids, primarily designed to fit around fingers or toes. The colored image FIG. 3 COLOR: ECO-SKIN (301-302) can depict how ECO-SKIN is applied to different parts of the body, illustrating its flexibility and adaptability in real-world scenarios. The contrast in color between the mannequin and ECO-SKIN allows for a better understanding of how the ECO-SKIN Strip (302) wraps around the fingers. It also highlights the semi-translucent/semi-permeable properties as seen with the color of the mannequin showing through the wrapped ECO-SKIN.

FIG. 4: FIG. 4 is the ECO-SKIN Wrap (401) configuration in action and is intended to function like a traditional gauze cotton wrap, designed to fit around circumferential body parts. The colored image FIG. 4 COLOR: ECO-SKIN (401-402) can depict how ECO-SKIN is applied to different parts of the body, illustrating its flexibility and adaptability in real-world scenarios. The contrast in color between the mannequin and ECO-SKIN allows for a better understanding of how the ECO-SKIN Wrap (401), wraps around the wrist of the mannequin. It also highlights the semi-translucent/semi-permeable properties as seen with the color of the mannequin showing through the wrapped ECO-SKIN.

FIG. 5: FIG. 5 is the ECO-SKIN Patch configuration in action. The ECO-SKIN Patch (501). is indented for placement directly on top of the wound site. The colored image FIG. 5 COLOR: ECO-SKIN (501-502) can depict how ECO-SKIN is applied to different parts of the body, illustrating its flexibility and adaptability in real-world scenarios. The contrast in color between the mannequin and ECO-SKIN allows for a better understanding of how the ECO-SKIN Patch (501), is placed around the forearm of the mannequin. It also highlights the semi-translucent/semi-permeable properties as seen with the color of the mannequin showing through the wrapped ECO-SKIN.

FIG. 6 COLOR: ECO-SKIN (601-603) helps to better understand the different ingredients of ECO-SKIN the components needed to grow ECO-SKIN. Without color, there's difficulty identifying the nuances of the quality of the ingredients. Additionally, color is needed when growing ECO-SKIN since you are constantly looking out for color changes in the fermented tea as well as the formation of the cellulose on the surface of the fermented tea

### Exemplary Ingredient 1. Green Tea

Traditionally green tea (602) is used in kombucha fermentation because it is less oxidized than other teas, which gives kombucha a lighter color and milder flavor. Additionally, green tea has powerful antimicrobial properties. These properties are due to green tea's rich content of polyphenols, particularly catechins, which are a type of antioxidant. These antimicrobial mechanisms of green tea disrupt microbial cell membranes, inhibiting enzyme activities essential for microbial growth, and interfering with the genetic material of pathogens. The most abundant catechin in green tea is epigallocatechin gallate (EGCG), which has been shown to inhibit the growth of various bacteria, viruses, and fungi (e.g., Streptococcus mutans), foodborne illnesses (e.g., Salmonella, E. coli), and hospital-acquired infections (e.g., Staphylococcus aureus, including MRSA).

### Exemplary Ingredient 2. Manuka Honey

Honey has been used for thousands of years, predominantly as a medical topical and an energy source for a wide range of organisms. Jun, as its commonly called, is green tea kombucha that uses honey as its primary glucose source. However, recent evidence has shown that Manuka honey (603), a specific honey that's produced by bees that pollinate the Manuka bush (Leptospermum scoparium) native to New Zealand and Australia, contains unique compounds, resulting in redound antimicrobial properties. Manuka honey has been shown to be effective against a wide range of bacteria, including both Gram-positive and Gram-negative species. It is particularly effective against antibiotic-resistant bacteria such as Methicillin-resistant Staphylococcus aureus (MRSA) and Pseudomonas aeruginosa. Methylglyoxal (MGO): The primary compound responsible for Manuka honey's antimicrobial activity is methylglyoxal. MGO is derived from the conversion of dihydroxyacetone (DHA), which is found in high concentrations in the nectar of Manuka flowers. MGO, at a concertation of at least 263 mg/kg has been shown to have potent antibacterial effects and is only found in Manuka honey, and no other honey on the planet. Additionally, Manuka honey also produces hydrogen peroxide, which has broad-spectrum antimicrobial properties. However, compared to other honeys that produce hydrogen peroxide, Manuka honey's high MGO content provides more consistent and stable antimicrobial properties. As a result, ECO-SKIN is grown using high MGO Manuka Honey or manuka honey with a UMF rating above 10. The UMF (Unique Manuka Factor) rating is a quality and grading system specifically for Manuka honey, indicating its antibacterial potency and authenticity. It measures the presence of key compounds like methylglyoxal (MGO), dihydroxyacetone (DHA), and leptosperin, which are unique to Manuka honey and contribute to its unique properties. As such, Researchers have found that Manuka honey had a higher antibacterial activity against various bacteria strains, with a stronger effect coming from the honey with higher UMF

### Exemplary Ingredient 3: (Scoby) Bi-Product of Eco-Skin Fermentation-Acidic Acid

Acetic Acid Bacteria (1604), commonly found in vinegar (Kombucha fermentation), is well-documented for its potent antimicrobial, anti-fungal, and anti-inflammatory properties. It effectively disrupts microbial cell membranes, leading to leakage of cellular contents and eventual cell death. It down this by significantly lowering the pH of its environment. Acetic acid creates conditions that are inhospitable for the growth of many pathogens, including common bacteria, fungi, and some viruses. This acidification also denatures essential proteins and enzymes within microbial cells, further inhibiting their metabolism and proliferation. Studies have been shown to support the use of acetic acid at a concentration of 1% to be used against various bacterial infections on the skin. As such, Acetic acid's broad-spectrum activity is particularly effective against both Gram-positive and Gram-negative bacteria, such as Staphylococcus aureus and Escherichia coli, as well as fungi like Candida albicans. Additionally, its ability to disrupt biofilms, which are protective layers formed by microbial communities, enhances its efficacy in treating infections, making it a valuable agent in wound care and infection control. The colored image FIG. 9 COLOR: ECO-SKIN (601-603) helps to better understand the different ingredients of ECO-SKIN. Without color, there's difficulty identifying the ingredients, specifically the green tea leave (602). Additionally, the color images highlight the natural beauty of the ingredients and how they come together to make ECO-SKIN.

FIG. 7: FIG. 7 is the precise flow chart fermentation process of growing, harvesting and post processing ECO-SKIN, modeled after traditional kombucha fermentation. FIG. 7 COLOR process flow enhances clarity by distinguishing each major stage in a different Color, aiding in the various steps, decision points, and procedural pathways. It provides a visual structure that helps facilitates accurate understanding of the sequence and relationship between process elements.

### Preparation of Manuka Honey Eco-Skin Wound Dressing

1. Brewing Green Tea: Begin by brewing organic green tea in distilled water at a concentration of 0.5% to 1.0% (w/v) and cooling the brewed tea solution to a temperature between 25° C. and 30° C. This temperature range is chosen to be the optimal temperature for the acetic acid bacteria. Any hotter 30°+ and it would kill the bacteria instantly and any cooler would reduce the activity of the bacteria. Green tea (602) is chosen for its polyphenolic compounds, particularly catechins like epigallocatechin gallate (EGCG), which have significant antimicrobial and antioxidant properties that support wound healing and infection prevention and provides a nutrient rich solution for the acetic acid bacteria to thrive in.

2. Adding Manuka Honey: Once the tea is brewed and cooled to room temperature, between 25° C. and 30° C., add medical-grade Manuka honey having a UMF rating of 10+ (902) at a concentration of 8% to 12% by weight/volume (w/v). Manuka honey serves as the primary glucose source necessary for the fermentation process. Furthermore, the concentration range is between 8-12% w/v depending on how thick the desired bacterial cellulose sheet is. If the desired bacterial cellulose sheet is greater than ¼^{th} thick, then a higher concentration of manuka honey is desired for prolonged fermentation. As such a thinner bacterial cellulose sheet is generated quicker and doesn't need the high concentration of manuka honey. However, going below 8% w/v proposes a risk of delayed fermentation where the yeast doesn't make enough alcohol, and the bacteria can't convert that alcohol into acids. Furthermore, too high concentration or greater than 12% w/v proposes a risk where the production of ethanol is increases and the bacteria cant convert it into acid. Therefore, the range of 8% to 12% by weight/volume (w/v) is put in place to provide flexibility within the fermentation process and provides a risk reduction.

3. Introducing Acetic Acid Bacteria: Once the manuka honey is fully dissolved, introduce an existing SCOBY starter culture (601) comprising of a concentration of 10% to 20% by weight/volume (w/v). This starter liquid provides a pre-acidified environment and an active microbial population that accelerates fermentation, promotes early pH reduction, and inhibits the growth of undesirable microorganisms. Concentrations below 10% may result in insufficient acidification and a slower fermentation process, while concentrations above 20% may inhibit yeast activity and alter the fermentation symbiosis. These bacteria in the starter culture are responsible for fermenting the sugars present in the mixture, producing acetic acid as a byproduct as well as bacterial cellulose at the air liquid interface.

4. Fermentation Process: The solution is fermented under static, aerobic conditions at a controlled temperature of 28° C.±2° C. This range is optimal for acetic acid bacteria growth; higher temperatures risk bacterial death, while lower temperatures may stall fermentation and promote mold formation. The process is conducted under semi-open-air conditions to ensure optimal oxygen availability at the air-liquid interface. During fermentation, the solution's pH decreases to between 3.5 and 4.0, corresponding to an acetic acid concentration of approximately 0.5% to 1.0% by volume. Under typical conditions, static fermentation proceeds for about 7-10 days, with pH monitored twice daily, until the target acidity is reached and the bacterial cellulose pellicle attains a thickness of approximately ¼^{th} in. During the fermentation process, the acidity of the solution will reach 3.4-4 before the pellicle is fully formed. As such, the pellicle will be harvested once all three are met: (1. pH=3.5-4) (2. Acetic acid concentration=0.5% to 1.0% by volume) and (pellicle reaches a thickness of approximately ¼ in). This will ensure that each batch is consistent for harvesting and for antibacterial properties.

5. Formation of bacterial cellulose: During the fermentation process, a Symbiotic Culture of Bacteria and Yeast (SCOBY)/bacterial cellulose forms at the air liquid interface. This bacterial cellulose sheet is the result of the fermentation process, incorporating the antimicrobial properties of the manuka honey and acetic acid.

6. Processing ECO-SKIN: Once the three criteria are met, the bacterial cellulose (BC) sheet is then carefully harvested from the fermentation solution. The BC sheet is then subjected to controlled dehydration under sterile conditions at a temperature between 30° C. and 40° C. for 5 to 7 hours. This drying process reduces the moisture content to approximately 10-12%, creating a dormant state in the embedded acetic acid bacterial colonies while preserving their viability for future reactivation upon hydration. Furthermore, temperature ranging 40° C.+ would kill the acetic acid bacteria present on the BC sheet, reducing the potential antibacterial properties. And the moisture content of 10-12% provides creates a “dormant state” in the embedded acetic acid bacterial colonies while preserving their viability for future reactivation upon hydration. This low moisture content prevents the bacteria from further propagating. This is further tested by a moisture analyzer to ensure that each patch is dehydrated to the proper moisture %. As such, the resulting dehydrated BC maintains both structural integrity and biological functionality, making it suitable for extended storage and later application.

7. Sterilization: For use cases where ECO-SKIN needs to be completely sterilized, there are various methods that can be used without damaging the structure or mechanical properties. Processes such as gamma irradiation, autoclaving (121-134° C. steam), or UV-C treatment (254 nm+), chosen based on application needs and material compatibility and desired application. Autoclaving will properly kill all bacterial colonies present on ECO-SKIN but risk damaging the mechanical properties. Gamma irradiation is another option, although expensive. UV-C treatment at a wavelength (nm) above 254 is a viable option due to its low cost, scalability,

7. The dehydrated ECO-SKIN sheet is subjected to precision shaping using a steel roll die cutter to achieve the required dimensions and form factor (201-204). Die cutting ensures consistent sizing tailored to the specific anatomical site or application, enabling efficient packaging, application, and clinical usability.

8. Packaging: The post fabricated pieces are then affixed with a release liner and then placed in airtight bags (1001-1003) to maintain sterility to prolong their shelf life until needed for wound dressing applications. The die-cut ECO-SKIN units are carefully affixed to a sterile, non-stick release liner. They are then vacuum-sealed in moisture-resistant packaging to maintain the target 10-12% moisture content, enabling long-term storage, sterile distribution, and optional functionality. For alternative configurations, skin safe/breathable adhesive backings will be applied to die cut ECO-SKIN (1301-1303) to increase the adhesive properties and allow for proper movement/lifestyle activities as well as packed in Dual Chamber Blister Activation Pack

FIG. 8: FIG. 8 illustrates the process of rehydrating ECO-SKIN using a dual-compartment activation pack. Inspired by the activation mechanism of a Dual Chamber Blister Activation Pack, the ECO-SKIN is rehydrated when the liquid compartment (801) is compressed, rupturing a frangible seal (803) and allowing 1-50 ml of rehydration solution (804) depending on the size of the dehydrated ECO-SKIN, to flow into the adjoining compartment containing the dehydrated ECO-SKIN sheet (802). This starts a transformative process, rehydrating the dressing and activating the dormant colonies of acetic acid bacteria embedded within the cellulose matrix. The term “activation” refers to the process of restoring the wound dressing from its dormant (dehydrated state) to its functional, hydrated state by application of the rehydration solution. Inspired by the preservation techniques used for freeze-dried probiotics, ECO-SKIN is dehydrated to maintain the viability of its beneficial microorganisms until reactivated with a measured volume of rehydration solution. The rehydration solution comprises the same aqueous fermented kombucha composition used to cultivate the bacterial cellulose during manufacture, containing green tea, medical-grade Manuka honey (UMF 10+ or greater), and beneficial acetic acid bacteria. Activation is considered complete when the dressing achieves a uniform moisture content of ≥90% by weight, typically within 1-2 minutes of rehydration solution contact.

While certain embodiments describe rehydrating ECO-SKIN with a fermented kombucha solution via a Dual Chamber Blister Activation Pack, other embodiments include rehydration solutions may include sterile saline, sterile water for general wound care; therapeutic formulations with antimicrobial agents (manuka honey/honey derivatives, iodine, acetic acid), regenerative agents (hyaluronic acid, collagen), or anti-inflammatory agents (corticosteroids, antibiotics) for targeted clinical use; cosmetic blends containing vitamins, antioxidants, or moisturizers for skin health and other cosmetic applications. In all cases, rehydration restores the bacterial cellulose matrix to a hydrated state with enhanced flexibility, tensile strength, adhesion, and the incorporation of the specific hydration solution. As such, the dual chamber blister activation method enables precise dosing from 1-50 ml depending on the size of the dehydrated bacterial cellulose configuration. Extended shelf stability is achieved by storing a pre-measured volume of rehydration solution in a sterile, vacuum sealed compartment physically separated from the dehydrated bacterial cellulose matrix. This configuration prevents premature activation, preserves material integrity, and ensures precise, uniform hydration upon use, while also creating a streamlined, single-step activation process for the user.

FIG. 9: FIG. 9 shows the step-by-step method (901) for preparing, activating, and applying ECO-SKIN using a Dual Chamber Blister Activation Pack. The process involves cleaning the application site, mechanically breaking the hydration fluid pouch to release a pre-measured rehydration solution into the dehydrated dressing, and opening the packaging to access the now-rehydrated ECO-SKIN. The hydrated dressing is applied directly to the wound, smoothed for full contact, worn for the recommended duration, and then removed and disposed of according to proper waste protocols. This method ensures precise liquid measurement, uniform activation, and user-friendly application while maintaining sterility and shelf stability. FIG. 9 COLOR enhances clarity by distinguishing each major stage in a different Color, aiding in the various steps, and user instructed pathways. It provides a visual structure that helps facilitates accurate understanding of the sequence and relationship between process elements.

FIG. 10: FIG. 10 shows three embodiments of the dehydrated ECO-SKIN wound dressing Wrap (1001), Patch (1002), and Strip (1003), each die-cut to specific sizes (2.6″×4″, 2″×2″, and 1″×3″) and then vacuum-sealed in sterile moisture-resistant packaging. The dehydrated bacterial cellulose sheet is preserved for later rehydration and use, with each format tailored for different anatomical applications and wound types. FIG. 10: COLOR in this figure accurately represents the appearance of the vacuum sealed ECO-SKIN within the sterile packaging, enabling clear identification of each variant's packaging. It provides a realistic visual reference that supports distinguishing between different product sizes and formats as well as visual indication of the health/status of the product.

FIG. 11: FIG. 11 visually shows a three-step process for applying ECO-SKIN without the need for rehydration solution or external adhesives. In Step 1 (1101), the user carefully removes the dressing from its sterile vacuum sealed packaging, ensuring not to touch the side that contacts the skin. In Step 2 (1102), the dressing is placed directly onto clean, affected area, where the natural stickiness of Manuka honey and porous nature of the bacterial cellulose helps it adhere to the skin. In Step 3 (1103), the dressing stays securely in place, that can be worn for up to 10 hours, depending on the wound type and advice from medical professional. FIG. 11: FIG. 11 COLOR showcases the natural auburn color of ECO-SKIN. ECO-SKIN is naturally semitransparent which allows for the user to look at the wound in real time and monitor any visual chances. In this instance, color is important to showcase the visual changes ECO-SKIN will go through, over the duration of use.

FIG. 12: FIG. 12 is a user sequence flowchart for applying ECO-SKIN without the need for rehydration solution or external adhesives (1201). The user first removes the dressing from its sterile packaging. A release liner is removed, then the dressing is applied directly onto clean, dry skin, where it naturally adheres due to the inherent tackiness of the bacterial cellulose and Manuka honey formulation. Once in place, the dressing forms a gentle seal without the need for external adhesives or rehydration solution and can remain on the skin for up to 10 hours, depending on the wound type and clinical context. The user then disposes of ECO-SKIN in the trash or compost. FIG. 12 COLOR enhances clarity by distinguishing each major stage in a different Color, aiding in the various steps, and user instructed pathways. It provides a visual structure that helps facilitates accurate understanding of the sequence and relationship between process elements.

FIG. 13: FIG. 13 shows three embodiments of the dehydrated ECO-SKIN wound dressings affixed to a biocompatible adhesive backing wrap (1301), Patch (1303), and Strip (1304), each die-cut to specific sizes (2.6″×4″, 2″×2″, and 1″×3″) and then vacuum-sealed in sterile moisture-resistant packaging. The dehydrated bacterial cellulose sheet is preserved for later use, with each format tailored for different anatomical applications and wound types. FIG. 13 COLOR showcase the auburn color of the ECO-SKIN and the semitransparent backing affixed to the back of it. The adhesive will need to be semitransparent, biocompatible and breathable for proper wound healing.

FIG. 14: FIG. 14 is a user sequence flowchart for applying ECO-SKIN without the need for a rehydration solution or external adhesives affixed to a biocompatible adhesive backing. (1401). The user first removes the dressing from its sterile packaging. A release liner is removed, then dressing is then applied directly onto clean, dry skin, where it naturally adheres due to the inherent tackiness of the bacterial cellulose and Manuka honey formulation as well as the adhesive backing. Once in place, the dressing forms a gentle seal without the need for external adhesives or rehydration solution and can remain on the skin for up to 10 hours, depending on the wound type and clinical context. Once complete, the user then disposes of ECO-SKIN in the trash or compost. FIG. 14 COLOR process flow enhances clarity by distinguishing each major stage in a different Color, aiding in the various steps, decision points, and procedural pathways. It provides a visual structure that helps facilitates accurate understanding of the sequence and relationship between process elements.

FIG. 15: FIG. 15 showcases the results of a Kirby-Bauer disk diffusion test using rehydrated ECO-SKIN. The goal of these tests was to evaluate ECO-SKINS inherent antibacterial properties against three strains of bacteria commonly found on chronic wound sites, and prone for antibiotic resistance. As such, the bacteria were: Staphylococcus aureus (1501), Pseudomonas fluorescens (1502), and Escherichia coli (1503). In each case, clear zones of inhibition formed around the ECO-SKIN sample, indicating its effectiveness at stopping bacterial growth. These visible “halos” demonstrate broad-spectrum antibacterial activity. FIG. 15 COLOR is needed to clearly highlight the contrast between bacterial growth, ECO-SKIN and the zones of inhibition. In this test case the dehydrated ECO-SKIN was hydrated using the fermented kombucha solution it was grown in, highlighting it's antibacterial properties and potential for treatment of chronic wounds.

FIG. 16: FIG. 16 are scanning electron microscope (SEM) images of dehydrated ECO-SKIN at varying levels of magnification: 100 μm at 500× (1601), 500 μm at 100× (1602), 50 μm at 1000× (1603), and 20 μm at 2000× (1604). These images reveal the porous matrix structure of the bacterial cellulose matrix, which provides both breathability and a water uptake capability for leaky wounds. Acetic acid bacteria are visibly integrated throughout the material (1604). Their presence highlights the intricate nature of ECO-SKIN and its composition. FIG. 16 COLOR is needed to enhance contrast and detail, allowing the fiber structure and bacterial features at each magnification level.

FIG. 17: FIG. 17 is a diagram explaining the ingredients, active components and bacterial strains of “ECO-SKIN.” It is divided into two main sections: “Ingredients” and “Active Compounds/Bacterial Strains.” The ingredients section shows that ECO-SKIN is composed of Green tea, rich in the antioxidant EGCG, protects the skin from free radical damage, soothes irritation, reduces redness, and regulates oil production, Manuka honey with its high (MGO) methylglyoxal content, offers potent antibacterial and anti-inflammatory properties, aiding in wound healing, preventing infections, and maintaining skin hydration and SCOBY (Symbiotic Culture of Bacteria and Yeast) introduces beneficial bacteria, including acetic acid bacteria (Gluconacetobacter) and lactic acid bacteria (Lactobacillus), which have powerful antimicrobial properties FIG. 13: COLOR ECO-SKIN (1701.) Adds richness to the information presented, but otherwise, isn't completely necessary when taking about the ingredients, active compounds and bacterial strains.

1. A reactivatable wound dressing composition comprising a dehydrated bacterial cellulose matrix derived from kombucha fermentation of organic green tea and Manuka honey, wherein the matrix comprises bacterial cellulose fibers having dormant colonies of acetic acid bacteria embedded therein, residual epigallocatechin gallate (EGCG) from the organic green tea, and residual methylglyoxal (MGO) from the Manuka honey, wherein the dormant colonies of bacteria are configured to reactivate upon contact with an aqueous rehydration solution to provide antimicrobial activity, and wherein the dehydrated matrix exhibits increased adhesive properties upon rehydration without requiring additional adhesives.

2. The wound dressing composition of claim 1, wherein the bacterial cellulose matrix has a moisture content of 10-12% in the dehydrated state.

3. The wound dressing composition of claim 1, wherein the acetic acid bacteria are selected from the group consisting of Komagataeibacter, Novacetimonas, Gluconacetobacter, Acetobacter, and mixtures thereof.

4. The wound dressing composition of claim 1, wherein the composition is die-cut into a shape selected from the group consisting of squares, rectangles, circles, ellipses, strips, and anatomically contoured shapes configured to conform to specific body regions, including but not limited to knees, elbows, heels, knuckles, and fingertips.

5. The wound dressing composition of claim 1, wherein the composition possesses a tensile strength sufficient to retain structural integrity in a hydrated state.

6. The wound dressing composition of claim 1, wherein the methylglyoxal concentration is provided by Manuka honey meeting medical-grade standards, the honey having a Unique Manuka Factor (UMF) rating of at least 10+, corresponding to a methylglyoxal (MGO) concentration of at least 263 mg/kg, and recognized Manuka honey certification programs.

7. A method for making a reactivatable bacterial cellulose wound dressing, comprising brewing organic green tea in distilled water at a concentration of 0.5% to 1.0% (w/v), cooling the brewed tea solution to a temperature between 25° C. and 30° C., adding medical-grade Manuka honey having a UMF rating of 10+ or greater to the cooled tea solution at a concentration of 8% to 12% by weight/volume, adding a SCOBY starter culture comprising fermented kombucha at a concentration of 10% to 20% by weight/volume, fermenting the mixture under static, aerobic conditions at 28° C.±2° C. Harvest when the bacterial cellulose (BC) concurrently satisfies three criteria: (1) pH 3.5-4.0, (2) acetic acid 0.5-1% and (3) thickness of the bacterial cellulose is approximately ¼^{th} in. Once these criteria are met, the bacterial cellulose is removed, dehydrated at a temperature of 30° C. to 40° C. for 5-7 hours to create dormant colonies of bacteria while maintaining their ability to reactivate upon subsequent hydration, wherein the dehydrated pellicle retains 10-12% moisture content. The resulting pellicle is die cut into specific shapes depending on the desired use case.

8. The method of claim 7, wherein the fermenting is continued until the acetic acid concentration reaches 0.5% to 1.0%.

9. The method of claim 7, further comprising sterilizing the pellicle prior to dehydration using a method selected from the group consisting of gamma irradiation, autoclaving (121-134° C. steam), and UV-C treatment with a 254 nm wavelength or greater.

10. The method of claim 7, wherein the dehydrating is performed under sterile conditions.

11. The method of claim 7, further comprising affixing the dehydrated pellicle to a biocompatible adhesive backing for enhanced securement.

12. The method of claim 7, further comprising vacuum sealing the dehydrated pellicle in sterile medical packaging.

13. The method of claim 7, wherein the dehydrated cellulose sheet is used without a rehydration solution and relies on the adhesive properties of the cellulose and honey for prolonged skin adhesion.

14. The method of claim 7, further comprising the dehydrated pellicle be placed on the skin through the tacky nature of the honey and bacterial cellulose.

15. A method for activating a dormant bacterial cellulose wound dressing, comprising providing a dehydrated bacterial cellulose wound dressing according to claim 1, rehydrating the dressing with a rehydration solution comprising the same fermented kombucha composition used to grow the bacterial cellulose matrix to rehydrate the cellulose fibers and reactivate the dormant colonies of bacteria embedded therein, and applying the reactivated dressing to a wound site, wherein the reactivation transforms the dressing from a dormant state to an activated state and generates adhesive properties through surface tension and residual Manuka honey tackiness.

16. A method for treating a wound, comprising providing a dehydrated bacterial cellulose wound dressing according to claim 1, rehydrating the dressing with a rehydration solution comprising fermented kombucha to rehydrate the bacterial cellulose matrix and reactivate dormant colonies of bacteria, and applying the rehydrated dressing to a wound site, wherein the dressing maintains a moist wound environment while providing antimicrobial protection.

17. The method of claim 15, wherein the rehydration solution has the same composition as the fermentation medium used to produce the bacterial cellulose matrix.

18. The method of claim 15, wherein the rehydrated dressing adheres to the skin without additional adhesives through surface tension and residual tackiness from Manuka honey.

19. The method of claim 15, wherein the wound is selected from the group consisting of cuts, burns, surgical incisions, chronic wounds, and skin ulcers.

20. The method of claim 15, wherein the dressing is left in place for up to 10 hours before replacement, unless otherwise directed by a medical professional.

21. The method of claim 15, wherein the antimicrobial protection is effective against bacteria selected from the group consisting of Staphylococcus aureus, Escherichia coli, and Pseudomonas aeruginosa.

22. A method for promoting wound healing, comprising applying the wound dressing composition of claim 1 to a wound site, wherein the bacterial cellulose matrix stimulates migration and proliferation of fibroblasts and endothelial cells, maintains optimal oxygen exchange while retaining moisture, provides a semi-permeable barrier against contaminants, and creates an acidic microenvironment that inhibits pathogenic bacterial growth.

23. The method of claim 21, wherein the wound healing is accelerated compared to conventional gauze dressings.

24. The method of claim 21, wherein the dressing reduces scarring formation.

25. A method for rehydrating a dehydrated bacterial cellulose wound dressing, comprising storing the dressing in a closed, dual-chamber, vacuum-sealed dual chamber blister pack containing a fixed, pre-measured quantity ranging from 1-50 ml of rehydration solution separated from the dressing by a breakable seal, wherein activation is achieved by rupturing the seal to release the solution and uniformly hydrate the dressing, the configuration providing precise liquid delivery, extended shelf life, and maintenance of sterility until use.

26. The method of claim 25 wherein the rehydration solution is selected from the group consisting of but not limited to fermented manuka honey kombucha solution, sterile saline or water, therapeutic agents (antimicrobials, regenerative compounds, anti-inflammatories), or cosmetic blends (vitamins, antioxidants, moisturizers).

27. A method for providing sustained antimicrobial activity at a wound site, comprising applying the wound dressing composition of claim 1 to the wound site, wherein the embedded acetic acid bacteria provide continuous antimicrobial activity through production of acetic acid and maintenance of an acidic pH environment hostile to pathogenic microorganisms.
